(19) United States
(12) Patent Application Publication
(10) Pub. No.: US 2020/0124567 A1
(43) Pub. Date: Apr. 23, 2020

(54) APPARATUS AND METHOD FOR DETERMINING WATER CHLORIDE CONCENTRATION

(71) Applicant: WALTRON BULL & ROBERTS, LLC, Flemington, NJ (US)

(72) Inventors: Uwe Gronowski, Berlin (DE); Eric Etter, Doylestown, PA (US)

(73) Assignee: WALTRON BULL & ROBERTS, LLC, Flemington, NJ (US)

(21) Appl. No.: 16/168,322

(22) Filed: Oct. 23, 2018

(51) Int. Cl. G01N 27/49; G01N 27/403

(52) U.S. Cl. CPC G01N 27/49; G01N 33/182; G01N 27/4035

(57) ABSTRACT

An apparatus for determining water chloride concentration is provided which comprises a chloride concentration measuring device, a power supply and a processor. The measuring device comprises a first portion configured to hold a reference solution. The first portion includes a reference electrode configured to contact the reference solution. The measuring device also comprises a second portion, adjacent the first portion, which is configured to receive sample water at a flow rate. The second portion includes a measuring electrode configured to contact the sample water. The processor is configured to control the power supply to apply a constant voltage across the reference electrode and the measuring electrode, receive an indication of a current, produced in the sample water in response to the constant voltage, which is proportional to an amount of chloride ions in the sample water and determine a chloride concentration of the sample water based on the current.

### SUMMARY

The present application discloses apparatuses and methods for detecting chloride concentration of water.

A chloride concentration measuring device is provided which includes a first portion configured to hold a reference solution. The first portion comprises a reference electrode configured to contact the reference solution. The measuring device also includes a second portion, adjacent the first portion, and configured to receive sample water at a flow rate. The second portion includes a measuring electrode configured to contact the sample water. When a constant voltage is received across the reference electrode and the measuring electrode, a current is produced in the sample water which is proportional to an amount of chloride ions in the sample water.

An apparatus for determining water chloride concentration is provided which comprises a chloride concentration measuring device, a power supply and a processor. The measuring device comprises a first portion configured to hold a reference solution. The first portion includes a reference electrode configured to contact the reference solution. The measuring device also comprises a second portion, adjacent the first portion, which is configured to receive sample water at a flow rate. The second portion includes a measuring electrode configured to contact the sample water. The processor is configured to control the power supply to apply a constant voltage across the reference electrode and the measuring electrode, receive an indication of a current, produced in the sample water in response to the constant voltage, which is proportional to an amount of chloride ions in the sample water and determine a chloride concentration of the sample water based on the current.

A method of detecting chloride concentration of water is provided which includes receiving, at a flow rate, sample water at a portion of a chloride concentration measuring device. The method also includes controlling a constant voltage to be applied across a measuring electrode, disposed in the portion and electrically connected to the sample water flowing in the portion and a reference electrode disposed in another portion of the chloride concentration measuring device which is adjacent the portion and holds a reference solution contacting the reference electrode. The method further includes determining a chloride concentration of the sample water based on a current in the sample water, produced in response to the constant voltage, the current being proportional to an amount of chloride ions in the sample water.

### BRIEF DESCRIPTION OF THE DRAWINGS

A more detailed understanding can be had from the following description, given by way of example in conjunction with the accompanying drawings wherein:

FIG. 1 is a block diagram of an example analyzer used to detect chloride concentration of water according to embodiments disclosed herein;

FIG. 2A is a perspective view of the example chloride concentration measuring device according to an embodiment;

FIG. 2B is a cross sectional view of the chloride concentration measuring device shown in FIG. 2A;

FIG. 3 is a block diagram of an example analyzer, including a flow meter, used to determine chloride concentration of sample water having a variable flow rate according to an embodiment;

FIG. 4 is a block diagram of an example analyzer, including a constant flow provider, used to determine chloride concentration of sample water having a constant flow rate according to an embodiment; and

FIG. 5 is a flow diagram illustrating an example method of determining chloride concentration of water according to embodiments described herein.

### DETAILED DESCRIPTION

Chloride ions (Cl—) are recognized as harmful contaminants in applications that require ultrapure water and steam systems, such as power plants. For example, relatively low levels of Chloride ions present in the steam have been shown to be directly related to stress corrosion cracking of high energy components in the low pressure sections of steam turbines. This corrosion can reduce the life of the components, such as turbines, and in some cases, can cause failure of the components. Efforts to prevent such failures have led to the formation of groups, such as EPRI (Electric Power Research Institute), ASTM (American Society of Testing Materials), ASME (American Society of Mechanical Engineers), ISO (International Standards Organization) and others to establish strict standards for water and steam contaminants. These efforts have been hampered, however, by the lack of reliable and efficient direct measurement equipment for real-time monitoring of chloride contamination in ultrapure water systems.

Conventional techniques for detecting chloride concentration of water include titration, ion chromatography, degassed conductivity and techniques using ion selective electrodes. For example, titration techniques involve incrementally adding a titration mixture (e.g., silver nitrate mixture), of a known quantity and volume, to water until an indicator has a reaction. These titration techniques do not have sufficient sensitivity, however, to measure chloride at low levels required by the standards.

Ion chromatography techniques include detecting ions based on their electrochemical charge in comparison to other analytes. Ion chromatography can detect chlorides at levels of 100 ppb or less. Ion chromatography has not, however, been adapted to power plant steam sampling systems. In addition, the equipment is very costly and difficult to maintain.

Degassed conductivity includes measuring the electrical conductivity of the sample after cations and conductive gases have been removed. The amount of chloride ions in ultrapure water systems is indirectly estimated. Degassed conductivity measures the total anions in water, however, which typically includes other anions such as sulfates, nitrates, amines, and other species. Accordingly, degassed conductivity techniques do not accurately measure the chloride concentration of the water.

Attempts to build chloride analyzers based on ion selective electrode techniques have been unsuccessful, due in part, because these analyzers are extremely complex, requiring precise temperature controls and buffering chemicals which are very toxic and difficult to use.

Some conventional techniques for detecting chloride concentration of water include measuring the concentration of another contaminant, such as sodium to indirectly measure the concentration of chlorides. For example, the level of sodium is proportional to the level of chlorides due to the common chemical pair of sodium chloride (i.e., table salt). There are, however, other more serious chemical species that can be present in water, such as HCl (hydrochloric acid), which can be present at low concentrations, even in an apparently alkaline environment.

The present application discloses apparatuses and methods for efficiently and accurately detecting low level amounts (e.g., <100 parts per billion (ppb)) of chloride concentration of water. The apparatuses and methods described herein provide an automated electrochemical process which determines the chloride concentration of the sample water in real time. That is, continuous and analogous readings (e.g., output current of the sample water passed by the counter electrode 124 to processor 116) of the chloride concentration are provided for the sample water as it flows through the measuring device. The readings are determined substantially instantaneously, with a signal constant buffering of up to about 20 seconds. The present application discloses cost efficient, easy to operate, low maintenance analyzers useful for monitoring steam and water systems in power plants.

An electrochemical process is used in which a constant voltage (i.e., voltage is maintained within a threshold voltage range) is applied between a reference electrode, which contacts a solution in a first portion of a measuring device, and a measuring electrode, which contacts sample water in a second portion of a measuring device. An output current is produced in response to the constant voltage. Based on the chemical reaction between the analyte (i.e., chloride) and the measuring electrode, as described below, the output current, which is proportional to the amount of chloride ions in the water, is determined. The chloride concentration of the water is measured according to the output current, the flow rate of the sample water and the temperature of the sample water.

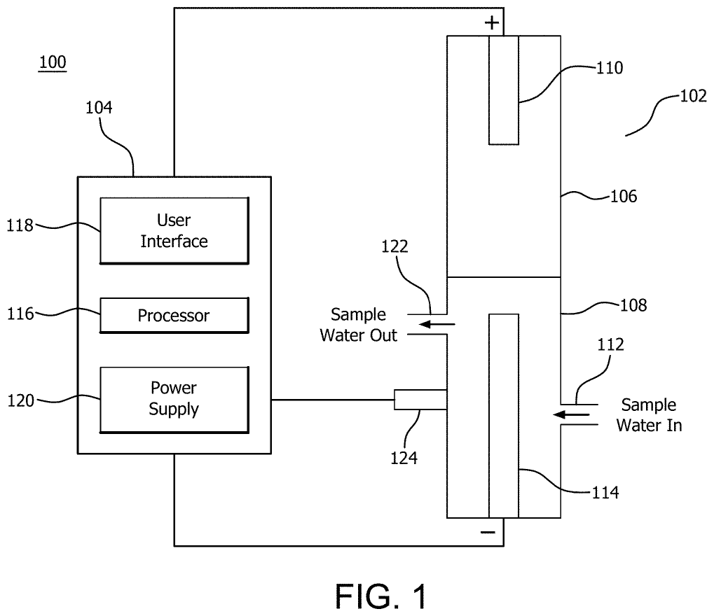

FIG. 1 is a block diagram of an example apparatus (i.e., analyzer) 100 used to detect chloride concentration in water according to embodiments disclosed herein. As shown in FIG. 1, the analyzer 100 includes chloride concentration measuring device 102 and electronics housing 104. The sizes, shapes and locations of each of the components shown in FIG. 1 are merely provided as an example.

The chloride concentration measuring device 102 includes a first portion 106 and a second portion 108, adjacent the first portion. The second portion 108 is, for example, disposed below the first portion 106 relative to ground during operation. The second portion 108 may not be disposed below the first portion 106, however, during operation. The first portion 106 is configured to hold a reference solution (e.g., a copper sulfate solution). The first portion 106 includes a reference electrode (e.g. copper) 110 configured to extend within the first portion 106 and contact the reference solution.

The second portion 108 is disposed below the first portion 106 relative to ground. The second portion 108 is configured to receive, via inlet 112, sample water at a flow rate. As described in more detail below, the sample water can be received at a constant flow rate or a variable flow rate. The second portion 108 also includes a measuring electrode 114 configured to extend within the second portion 108 and contact the sample water that is received via inlet 112. A surface of the measuring electrode 114 comprises a material reactive with chloride such that a measurable current is produced when an electric potential is applied. For example, the surface of measuring electrode 114 may include silver (e.g., silver chloride (AgCl)).

Electronics housing 104 is configured to house electronic and processing components used to control operation of the components and to communicate between the components described herein. Electronic and processing components include, for example, one or more processors 116 (hereinafter “processor”), user interface 118, power supply 120 and other components (not shown) such as memory (e.g., RAM), storage (e.g., removable storage device) circuitry, wires, buses, transmitters, receivers and network interfaces. The electronic and processing components may be configured to communicate (wired or wirelessly) with components of analyzer 100. Additionally one or more electronic and processing components, such as one or more additional control processors (not shown), can be located at one or more of the components of the analyzer 100 and configured to communicate with the electronic and processing components housed at electronics housing 104. The processor 116 is configured to process instructions (e.g., from user input and predefined programmed instructions).

As shown in FIG. 1, user interface 118 (e.g., touch screen display) is disposed at electronics housing 104. User interface 118 may be disposed at a location separate from electronics housing. User interface 118 is used to display operating conditions (e.g., chloride concentration measurements, sample water flow rate and sample water temperature) of analyzer 100. User interface 118 is also configured to receive user input for selecting parameters (e.g., numerical values) for setting operating conditions of components of the analyzer.

Power supply 120 is used to apply a constant voltage (i.e., electric potential) across the reference electrode 110 and the measuring electrode 114. For example, the processor 116 controls the power supply 120 to maintain a constant voltage within a target voltage range (e.g., about 130 mV to about 250 mV) such that the working potential of the measuring electrode 114 is reached under the following two conditions: 1) in the absence of chloride ions the counting electrode reads zero; and 2) linearity is achieved over the target measuring range and the reading from the counting electrode.

As described in more detail below, when the constant voltage is applied, an output current is produced, via counter electrode 124, based on the electrochemical half reaction between the material (e.g., silver chloride) on the surface of the measuring electrode 114 and the chlorides in the sample water. The output current is proportional to the amount of chloride ions in the water. The proportion is determined according to the temperature of the sample water, the surface area of the measuring electrode 114 and the flow rate of the sample water. The output current of the sample water flowing, via outlet 122 from the second portion 108, is provided by the counter electrode 124 to the processor 116. The processor 116 determines the chloride concentration based on the output current.

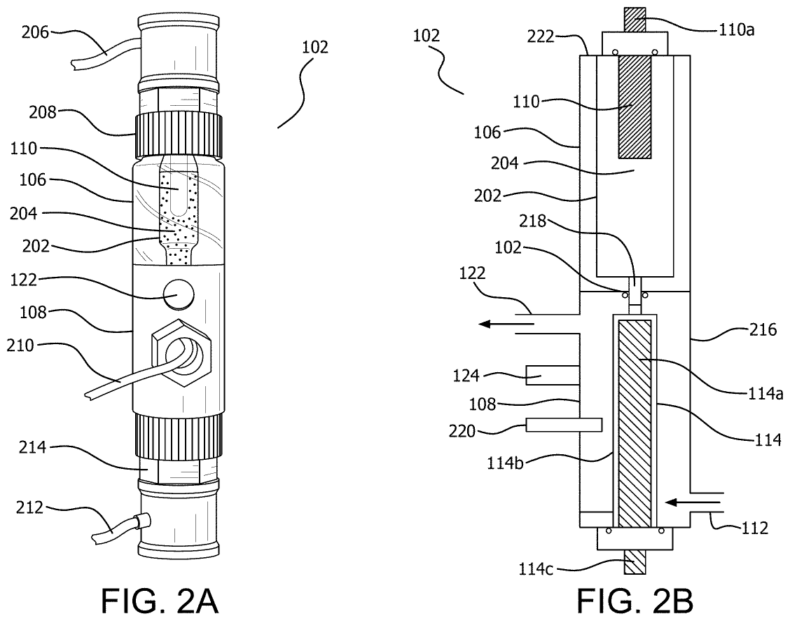

FIG. 2A is a perspective view of the example chloride concentration measuring device 102 according to an embodiment. FIG. 2B is a cross sectional view of the chloride concentration measuring device 102 shown in FIG. 2A. Referring to FIGS. 2A and 2B, first portion 106 includes a solution chamber 202 which includes a reference solution 204. The reference electrode 110 is disposed within reference solution 204. Reference electrode 110 is electrically connected to power supply 120 via electrically conductive material housed by wire 206. The wire 206 enters upper sealing assembly 208 and is electrically connected to a first portion 110a of reference electrode 110. Sealing assembly 208 includes, for example, a gasket (e.g., an O-ring) in the form of a ring with a circular cross section. The reference solution 204 and reference electrode 110 include material such that the constant voltage is applied across the measuring device without passing any current. Examples of materials of the reference solution and electrode pair include, but are not limited to, copper and copper-sulfate, silver and silver-chloride, mercury and mercury-sulfate, lead and lead-sulfate, palladium and palladium-sulfate, and calomel.

Second portion 108 includes a wall 216 (e.g., comprising stainless steel or another conductive metal material) defining a sample water flow chamber configured to hold the sample water flowing within. Second portion 108 includes sample water inlet 112, configured to receive the sample water into the flow chamber, and sample water outlet 122, configured to provide the sample water from the sample water flow chamber of the second portion 108.

Second portion 108 also includes measuring electrode 114, comprising a measuring electrode base 114a (e.g., comprising silver) and a measuring electrode surface (plating) 114b. The measuring electrode surface 114b is disposed within the second portion 108 such that the surface 114b contacts the sample water flowing through the second portion 108. Measuring electrode surface 114b comprises a material (e.g., silver chloride) configured to react with the chlorides in the sample water and produce a current in the sample water. The measuring electrode also includes a protective material comprising an electrochemically inert, hydrophobic elastic, for example acetate, disposed on the electrode surface 114b to extend the life of the electrode surface 114b. Measuring electrode 114 is electrically connected to power supply 120 via electrically conductive material housed by wire 212. The wire 212 enters lower sealing assembly 214 and is electrically connected to a second portion 114c of measuring electrode 114.

The chloride concentration measuring device 102 also includes diaphragm 218. As shown in FIG. 2B, the diaphragm 218 extends between the solution chamber 202 of the first portion 106 and the measuring electrode 114 providing for the measurement of the electric potential applied across the reference electrode 110 and the measuring electrode 114, while physically separating the sample water from the reference solution. Diaphragm 218 includes any material sufficiently porous to allow for the voltage to cross without draining the reference electrode or interacting with any of the electro-chemical elements, such as for example, ceramic aluminum. As shown in FIG. 2B, a gasket 222 (e.g., an O-ring) in the form of a ring is also disposed between the first portion 106 and the second portion 108.

Counter electrode 124 is electrically connected to the sample water (e.g., directly connected to the water or indirectly connected to the water via a part of the second portion (e.g., to the metal wall defining the second portion 108). Counter electrode 114 is also electrically connected to power supply 120 via electrically conductive material housed by wire 210. Counter electrode 124 is configured to receive an output current resulting from a half reaction of the chlorides in the sample water and, for example, the silver of the measuring electrode surface 114b. An indication (e.g., amperage value) of the output current is provided from the counter electrode 124 to processor 116. The processor 116 receives an indication of the output current and determines the chloride concentration of the sample water based on the output current.

The determination of the chloride concentration of the sample water is also based on the temperature of the sample water flowing through the measuring device 102. The processor 116 may scale the output current derived from the reaction according to a sensed temperature.

For example, as shown in FIG. 2B, the measuring device 102 includes a temperature sensor 220 disposed at the second section 108. The location of the temperature sensor shown in FIG. 2B is merely by way of example. Analyzer 100 may include a temperature sensor at any location along the water pathway such that the sensed temperature of the sample water, being measured in the measuring device 102 and flowing out of outlet 122, is accurate.

A temperature of the sample water is determined from a temperature sensor (e.g., temperature sensor 220 in FIG. 2B or another temperature sensor at a location along the water pathway between the flow meter 302 and the sample water outlet 122). The sensed temperature is provided to the processor 116, which stores the temperature (e.g., in memory or a storage device, not shown). The processor 116 may then scale the output current derived from the reaction according to the sensed temperature.

Alternatively, a constant temperature provider, such as a heat exchanger (not shown), which controls the temperature of the sample water provided to the measuring device 102 can be used to provide the sample water at a predetermined constant temperature (e.g., a temperature within a predetermined temperature range). Accordingly, the processor 116 may then determine the chloride concentration without scaling the output current according to the predetermined temperature.

The determination of the chloride concentration of the sample water is also based on the flow rate of the sample water flowing through the measuring device 102. The sample water may flow through the measuring device 102 at a constant rate or a variable rate. When the flow rate is variable, the processor 116 may scale the output current derived from the reaction according to a determined flow rate. Alternatively, the processor 116 determines the chloride concentration of the sample water according to a constant flow rate.

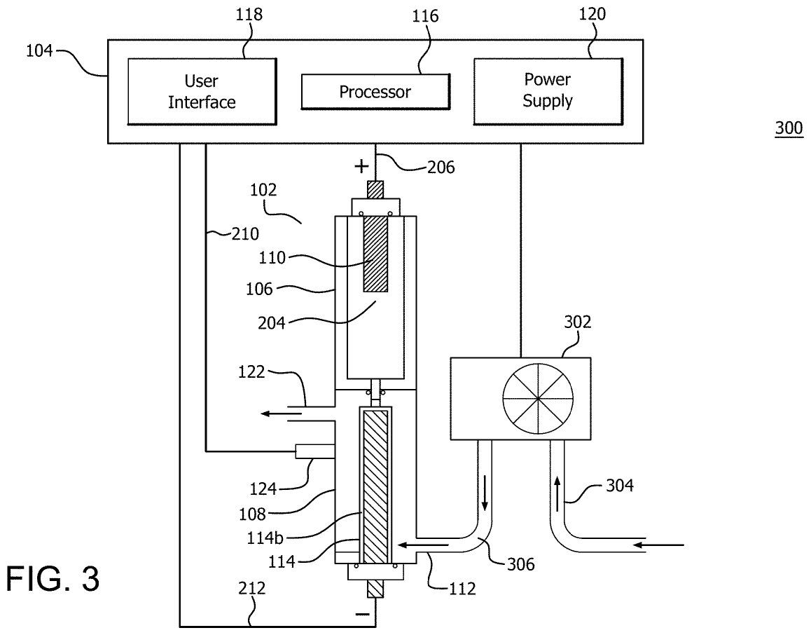

For example, FIG. 3 is a block diagram of an example apparatus 300, including a flow meter, used to determine chloride concentration of sample water having a variable flow rate according to an embodiment. As shown in FIG. 3, the analyzer 300 includes measuring device 102 and the components (e.g., processor 116, user interface 118, and power supply 120) of electronics housing 104 shown in FIGS. 1, 2A and 2B. The analyzer 300 also includes flowmeter 302. The flowmeter 302 includes, for example, one or more sensors used to determine a flow of the sample water over time.

Arrows are used in FIG. 3 to illustrate the flow of sample water through the components of the analyzer 300. The sample water is received at inlet conduit 304 and is provided to the flow meter 302. The flow meter 302 senses flow amounts of the sample water over time. The processor 116 determines a flow rate (e.g., amount of water per hour) of the sample water based on flow amounts of the sample water sensed over time. The processor 116 receives an indication (e.g., digital representation or analog representation) of the flow rate and stores (e.g., in memory or a storage device, not shown) the flow rate of the sample water.

The sample water flows from the flow meter 302 through outlet conduit 306 to the measuring device 102. The sample water is received into the second portion 108 of the measuring device 102 via sample water inlet 112. The sample water flows through the second portion 108 and exits the second portion 108 at water outlet 122.

Processor 116 controls a constant voltage to be applied across the measuring electrode 114 and the reference electrode 110. When the water contacts the measuring electrode surface 114b at the applied voltage, the material on measuring electrode surface 114b reacts with the chlorides in the sample water and produces a current in the sample water. For example, if the material on measuring electrode surface 114b is silver chloride, a reaction between the silver chloride and the chlorides in the sample water at the applied voltage produces the current in the sample water, as shown below in the half reaction equation.

Cl⁻+Ag→AgCl+e⁻ Equation (1)

The current in the sample water is passed by the counter electrode 124 to the processor 116. In response to receiving the indication of the current, the processor 116 determines an unscaled chloride concentration (e.g., a ratio of the chlorides to the sample water) of the sample water according to the current, which is proportional to the amount of chloride ions in the water. The proportion is calculable from the temperature of the sample and the flow rate. The processor 116 scales the determined unscaled chloride concentration of the sample water according to the determined flow rate.

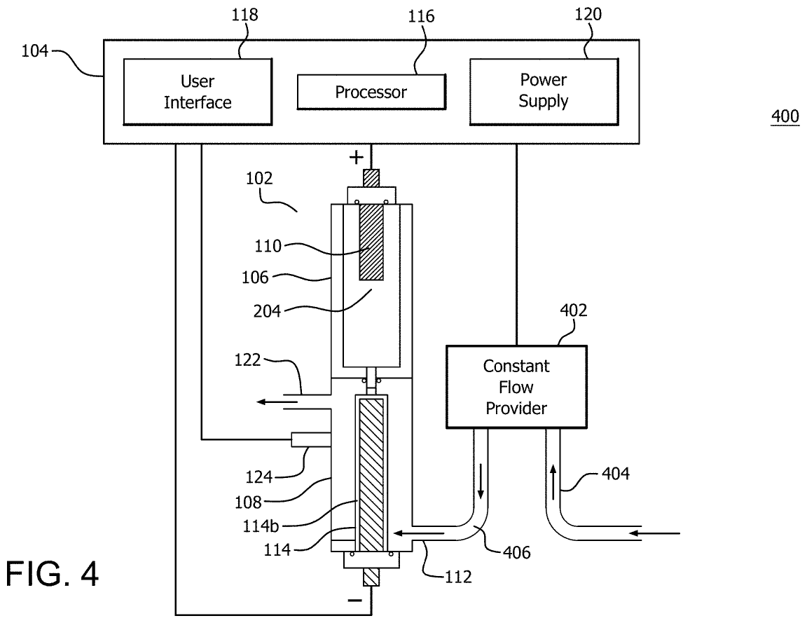

Alternatively, the flow rate of the sample water can be controlled such that sample water flows into the measuring device 102 at a constant rate. For example, FIG. 4 is a block diagram of an example analyzer, including a constant flow provider, used to determine chloride concentration of sample water having a constant flow rate according to an embodiment. Arrows are used in FIG. 4 to illustrate the flow of sample water through the components of the analyzer 400.

As shown in FIG. 4, the analyzer 400 includes measuring device 102 and the components (e.g., processor 116, user interface 118, and power supply 120) of electronics housing 104 shown in FIGS. 1, 2A, 2B and 3. The analyzer 300 also includes a constant flow provider 402 which receives sample water at a flow rate and provides the sample water to the second portion 108 of the measuring device 102 at a constant rate. For example, the constant flow provider 402 is a constant flow head which maintains the flow rate at a constant flow rate.

The sample water is received at inlet conduit 404 and is provided to the constant flow provider 402. The sample water flows from the constant flow provider 402 through outlet conduit 406 to the measuring device 102. The sample water is received into the second portion 108 of the measuring device 102 via sample water inlet 112. The sample water flows through the second portion 108 and exits the second portion 108 at water outlet 122.

As described above in FIG. 3, the processor 116 determines an unscaled chloride concentration (e.g., a ratio of the chlorides to the sample water) of the sample water according to the output current. The processor 116 scales the determined unscaled chloride concentration of the sample water according to the determined flow rate. Because the flow rate is provided to the measuring device 102 at a predetermined constant rate, however, the chloride concentration of the sample water is scaled by a flow rate constant.

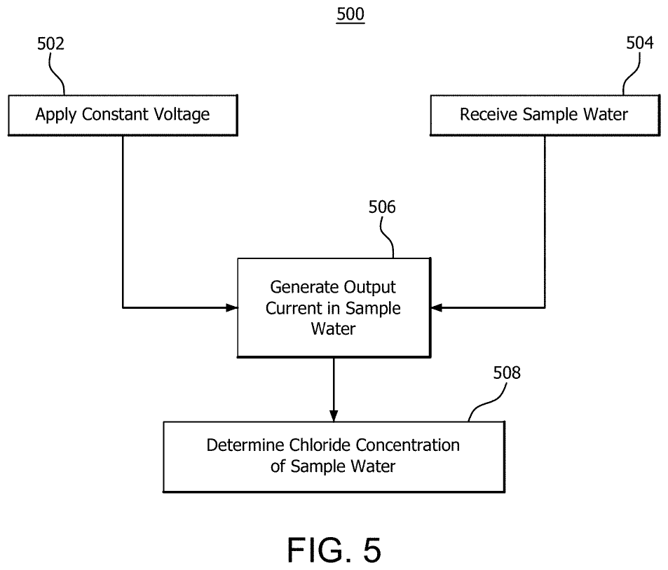

FIG. 5 is a flow chart illustrating an example method 500 of detecting chloride concentration. As shown at block 502 of FIG. 5, the method 500 includes applying a constant voltage across the measuring electrode in the second portion of the measuring device and the reference electrode in the first portion of the measuring device. For example, a processor is used to control a constant voltage to be maintained within a voltage range across the measuring electrode and the reference electrode.

As shown at block 504 of FIG. 5, the method 500 includes receiving sample water. For example, the sample water is received at a second portion of a chloride concentration measuring device. The sample water is provided to the measuring device simultaneous with applying the constant voltage, prior to applying the constant voltage (e.g., at start up or restart, the sample water is in the measuring device prior to applying the voltage) or after applying the constant voltage (e.g., the sample water is temporarily blocked while the voltage is still being applied). Accurate readings of the chloride concentration are provided, however, when the sample water is flowing in the measuring device while the constant voltage is applied.

As shown at block 506 of FIG. 5, the method 500 includes generating an output current in the sample water. For example, when the constant voltage is applied, an output current is produced based on the electrochemical half reaction between silver (e.g., silver chloride) on a surface of the measuring electrode and the chlorides in the water sample.

As shown at block 508 of FIG. 5, the method 500 includes determining a chloride concentration of the sample water based on the current. For example, the output current is passed to the processor via a counter electrode coupled to the measuring device. The processor then determines the chloride concentration of the sample water based on the current, which is proportional to an amount of chloride ions in the sample water.

In one embodiment, determining the chloride concentration of the sample water also includes determining an unscaled chloride concentration and scaling the chloride concentration based on a sensed variable temperature.

In another embodiment, determining the chloride concentration of the sample water also includes determining an unscaled chloride concentration and scaling the chloride concentration based on a determined variable flow rate.

The methods provided can be implemented in a general purpose computer, a processor, or a processor core. Suitable processors include, by way of example, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), and/or a state machine. Such processors can be manufactured by configuring a manufacturing process using the results of processed hardware description language (HDL) instructions and other intermediary data including netlists (such instructions capable of being stored on a computer readable media). The results of such processing can be maskworks that are then used in a semiconductor manufacturing process to manufacture a processor which implements features of the disclosure.

The methods or flow charts provided herein can be implemented in a computer program, software, or firmware incorporated in a non-transitory computer-readable storage medium for execution by a general purpose computer or a processor. Examples of non-transitory computer-readable storage mediums include a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

It should be understood that many variations are possible based on the disclosure herein. Although features and elements are described above in particular combinations, each feature or element can be used alone without the other features and elements or in various combinations with or without other features and elements.

What is claimed is:

1. A chloride concentration measuring device comprising:
a first portion configured to hold a reference solution and comprising a reference electrode configured to contact the reference solution; and
a second portion adjacent the first portion and configured to receive sample water at a flow rate, the second portion comprising a measuring electrode configured to contact the sample water,
wherein when a constant voltage is received across the reference electrode and the measuring electrode, a current is produced in the sample water which is proportional to an amount of chloride ions in the sample water.

2. The measuring device of claim 1, wherein the second portion comprises a wall formed of a conductive material and the measuring device further comprises a counter electrode, electrically coupled to the wall, configured to pass the current in the sample water from the measuring device.

3. The measuring device of claim 1, wherein the second portion further comprises a temperature sensor coupled to the second portion and configured to sense a temperature of the water in the second portion.

4. The measuring device of claim 1, wherein the reference solution comprises material which allows the constant voltage to be applied across the measuring electrode and the reference electrode without passing any current.

5. The measuring device of claim 1, wherein a surface of the measuring electrode comprises material which causes the current to be produced in the sample water based on a reaction of the material with chlorides in the sample water.

6. The measuring device of claim 5, wherein a protective material comprising of an electrochemically inert, hydrophobic plastic, such as acetate, is disposed on the surface of the measuring electrode.

7. The measuring device of claim 1, wherein the constant voltage is maintained in a range of about 130 mV to about 250 mV.

8. The measuring device of claim 1, further comprising a diaphragm extending between the reference solution in the first portion and the second portion, the diaphragm configured to communicate the constant voltage between the reference electrode and the measuring electrode and physically separate the sample water from the reference solution.

9. An apparatus for determining water chloride concentration, the apparatus comprising:
a chloride concentration measuring device comprising:
a first portion configured to hold a reference solution and comprising a reference electrode configured to contact the reference solution; and
a second portion adjacent the first portion and configured to receive sample water at a flow rate, the second portion comprising a measuring electrode configured to contact the sample water;
a power supply; and
a processor configured to:
control the power supply to apply a constant voltage across the reference electrode and the measuring electrode;
receive an indication of a current, produced in the sample water in response to the constant voltage, which is proportional to an amount of chloride ions in the sample water; and
determine a chloride concentration of the sample water based on the current.

10. The apparatus of claim 9, further comprising a temperature sensor coupled to the second portion and configured to sense a temperature of the sample water in the second portion,
wherein the processor is further configured to determine the chloride concentration of the sample water based on the sensed temperature.

11. The apparatus of claim 9, further comprising a temperature sensor disposed along a sample water pathway and configured to sense a temperature of the sample water in the second portion, and
wherein the processor is further configured to determine the chloride concentration of the sample water based on the sensed temperature.

12. The apparatus of claim 9, further comprising a flow meter coupled to a sample water inlet of the measuring device, the flow meter configured to:
receive the sample water; and
sense flow amounts of the sample water over time, and wherein the processor is further configured to:
calculate the flow rate of the sample water from the flow amounts sensed over time; and
determine the chloride concentration of the sample water based on the flow rate.

13. The apparatus of claim 9, further comprising a constant flow provider coupled to the second portion of the measuring device and configured to provide the sample water to the measuring device at a constant flow rate.

14. The apparatus of claim 9, further comprising a counter electrode configured to pass the current in the sample water from the measuring device to the processor.

15. The apparatus of claim 9, wherein the reference electrode and solution comprise materials which allow a constant voltage to be applied across the measuring electrode and the reference electrode without passing any current.

16. The apparatus of claim 8, wherein a surface of the measuring electrode comprises material which causes the current to be produced in the sample water based on a reaction of the material with chlorides in the sample water.

17. The apparatus of claim 16, wherein the material of the surface of the measuring electrode comprises silver chloride.

18. The apparatus of claim 9, further comprising a diaphragm extending between the reference solution in the first portion and the second portion, the diaphragm configured to communicate the constant voltage between the reference electrode and the measuring electrode and physically separate the sample water from the reference solution.

19. A method of determining water chloride concentration, the method comprising:
receiving, at a flow rate, sample water at a portion of a chloride concentration measuring device;
controlling a constant voltage to be applied across:
a measuring electrode, disposed in the portion and electrically connected to the sample water flowing in the portion, and
a reference electrode disposed in another portion of the chloride concentration measuring device which is adjacent the portion and holds a reference solution contacting the reference electrode; and
determining a chloride concentration of the sample water based on a current in the sample water, produced in response to the constant voltage, the current being proportional to an amount of chloride ions in the sample water.

20. The method of claim 19, wherein the constant voltage is applied simultaneous with the receiving of the sample water at the portion of the chloride concentration measuring device.
